(19) United States
(12) Patent Application Publication
(10) Pub. No.: US 2004/0235000 A1
(43) Pub. Date: Nov. 25, 2004

(54) Method for retrospective birth dating of biomolecules, cells, tissues, organs and organisms

(76) Inventors: Kirsty Spalding, Stockholm (SE); Jonas Frisen, Stockholm (SE)

Correspondence Address: MINTZ LEVIN COHN FERRIS GLOVSKY & POPEO, 666 THIRD AVENUE, NEW YORK, NY, 10017, US

(21) Appl. No.: 10/654,669

(22) Filed: Sep. 3, 2003

Related U.S. Application Data
(60) Provisional application No. 60/407,863, filed on Sep. 3, 2002.

(51) Int. Cl. C12Q 1/68

(52) U.S. Cl. 435/6

(57) ABSTRACT

This invention provides novel methods for the determining the birth dates or synthesis dates of biomolecules, organisms, cells, tissues and organs. Methods for determining the birth date of an organism by determining the birth date of a biomolecule of the organism are also provided.

### CROSS REFERENCE TO RELATED APPLICATIONS

This Application claims the benefit of priority from U.S. Ser. No. 60/407,863, entitled A Method for Retrospective Birth Dating of Cells” filed Sep. 3, 2002. All patents, patent applications, and references cited in this specification are hereby incorporated by reference, in their entireties.

### FIELD OF THE INVENTION

The invention provides novel methods for determining the age or birth date of biomolecules, cells, tissues and organs from animals, plants, viruses, as well as organism age.

### BACKGROUND OF THE INVENTION

Many cells in the body have a set life span. In some organs, cells undergo continuous turnover, and old cells are replaced by new ones. Often, mature or differentiated cells can divide to give rise to more cells of the same type. Yet, differentiated cell types such as neurons are unable to divide. In such cases, new cells are generated by less differentiated stem or progenitor cells. Information about cell turnover is crucial to the understanding of basic biological processes. Many diseases affect the generation of new cells, and information about cell turnover would provide novel insights into the causes and treatments for such diseases.

Cell turnover has been studied by several methods. One method evaluates cell markers that are selectively expressed in cells undergoing mitosis. The expression of such markers can be used to study cell proliferation for various tissues. Yet, this method provides a limited amount of information, since it only detects cells at the time of cell division. Because many cells die shortly after division, mitosis markers cannot provide an accurate assessment of the number of new cells that are formed in an organ or tissue. Moreover, this method cannot be used to measure the phenotype of new cells generated from stem or progenitor cells.

Another method involves labeling dividing cells with a stable, inheritable marker. This can be done by administering labeled nucleotides that integrate into the genome of dividing cells. Nucleotides can be labeled with a radioactive isotope (e.g., ³H-thymidine), which can be detected by autoradiography or other techniques. Nucleotides can also be labeled with a chemical modification (e.g. BrdU, a thymidine analogue), which allows detection by immunohistochemistry. Whilst useful, this method has several serious shortcomings. First, it does not provide a comprehensive picture of cell turnover, but only a snapshot taken at the time the nucleotide analogue is added. Second, modified nucleotides are toxic to dividing cells, resulting in a significant underestimate of actual cell turnover (Zhao, M., et al., (2003) Proc. Natl. Acad. Sci. USA 100(13):7925-7930). Third, because of this toxicity, modified nucleotides cannot normally be used for human studies. Fourth, since the nucleotide analogues must be administered to the organism when the cells are dividing, postmortem analysis is precluded.

Yet another method involves injecting retroviruses that have been constructed to express a particular marker. With this approach, only cells that complete a new cell division will take up the retrovirus. Although this method allows accurate determination of cell division, injection of the viral vectors causes trauma to the tissue surrounding the injection site, and cannot be used for human studies. Therefore, there is a need to develop more accurate methods for evaluating cell turnover, which can be used for post-mortem analysis, and for analysis of human organs and tissues.

The brain and the spinal cord have historically been considered regions devoid of neurogenesis in the periods following embryonic and early postnatal development. However, it has recently been established that new neurons are continuously generated from stem cells residing in the adult mammalian brain (McKay, 1997). Neurogenesis has been shown to occur in the song system and hippocampal formation of song-birds (Macklis), and new neurons have been found in the hippocampal formation and olfactory bulb of rodents (Altman and Das, 1965; Palmer, T. D., et al., (1997) Mol. Cell. Neurosci. 8:389-404; Johansson, C. B., et al., (1999) Cell 96:25-34). Neurogenesis has also been reported in primates in the dentate gyrus of the hippocampus and the subventricular zone lining the wall of the lateral ventricle (Gould, E., et al., (1999) Proc. Natl. Acad. Sci. USA 96:5263-5267).

Whether neurogenesis takes place in other regions of the adult primate brain remains controversial (Gould, E., et al., (1999) Science 286:548-552; Bernier et al., 2002; Koketsu et al., 2003; for review see Rakic, P., (2002) Nature Reviews 3:65-71). The majority of evidence for adult mammalian neurogenesis has been obtained from mouse and primate animal studies. In a single study, researchers analyzed brain tissue from patients who had received BrdU as part of a clinical trial for terminal larynx and tongue cancer (Eriksson, P. S., et al., (1998) Nature Medicine 4(11):1313-1317). Although the BrdU treatment regime was not optimized for detection of neurogenesis, the authors identified BrdU-positive cells in the dentate gyrns of the hippocampus and the subventricular zone of the lateral ventricle, with BrdU+cells of the dentate gyrns also positve for the neuronal maker NeuN. Additional studies performed in primate animals have suggested that adult neurogenesis also occurs in the amygdala and cortex (Gould, E., et al., (1999) Science 286:548-552; Bernier et al., 2002).

Carbon exists in the atmosphere in three isoforms: ¹²C, ¹³C and ¹⁴C. At low levels, ¹⁴C is produced in the atmosphere by the interactions of cosmic rays with nitrogen. However, atmospheric levels of ¹⁴C have been dramatically increased as a result of thermonuclear testing in the mid 1960's (Nydal and Lövseth, 1997). Thermonuclear testing ended in 1963 after the Test Ban Treaty came into force. Since that time, atmospheric levels of ¹⁴C have been exponentially decreasing, and today's levels are about 110% of the levels measured in 1950. The atmospheric ¹⁴C concentration is expected to reach pre-testing levels within the next 20 years (Lovell et al., 2002).

This exponential decrease in atmospheric ¹⁴C is due to an exchange of CO₂ in environmental reservoirs and the introduction of ¹⁴C depleted CO₂ from burning fossil fuels. It is not due to decay of ¹⁴C to other carbon species, since the ¹⁴C half-life is 5730 years. Atmospheric ¹⁴C levels have been measured by several researchers around the world and plotted as a function of delta ¹⁴C (atmospheric ¹⁴C measurements corrected for isotopic fractionation and radioactive decay) and time. This is used to create a bomb-spike plot. The plot shows sharp peaks in trophosphere radiocarbon in the early 1960s in the Northern Hemisphere, reflecting the location of most atomic weapons tests. Traditionally, ¹⁴C measurements have been made by counting the radioactive decay of individual carbon atoms. This technique is relatively insensitive and subject to statistical errors. The half-life of ¹⁴C is extremely long, allowing very few atoms to decay during the measurement period. In addition, living material contains only 100 attomoles of ¹⁴C per mg carbon, and DNA contains approximately 23-30% carbon mass. Thus, older detection methods cannot be used to resolve time differences of a few years.

Recent advances in the sensitivity and accuracy of mass-spectrometric techniques (e.g., accelerator mass spectrometry; AMS) have allowed counts for the number of ¹⁴C atoms in a sample to the level of parts per billion to parts per quadrillion (1×10⁹-1×10¹⁵). This can be used to date items to specific years rather than hundreds or thousands of years. AMS has been used to date biological samples, such as bone, gallstones (Mok et al., 1986), and senile plaques and neurofibrillary tangles in Alzheimer's disease (Lovell et al., 2002). Until now, no researchers have demonstrated the use of AMS for dating cells by measuring levels of ¹⁴C in DNA.

### BRIEF SUMMARY OF THE INVENTION

In accordance with this invention, AMS-based methods are used to measure the ¹⁴C of DNA and thereby to date (i.e., determine the age) of cells. After a cell has terminally differentiated it does not divide again. Because the last cell division represents the last time point when the cell synthesized DNA, its chromosomal DNA will reflect the date that the cell was produced. Therefore, establishing the age of ¹⁴C in chromosomal DNA allows determination of the “birth date” of cells and the rate of cell turnover. Advantageously, the methods of the invention can be used to investigate novel cell division and aberrations of cell division associated with disease, injury, or degenerative disorders. Cell divisions that could be study include CNS cell division (neurogenesis), and cell divisions in the liver, heart, and pancreas. In other aspects, the methods of the invention can be used for birth dating cells, tissues, organs, and organisms such as humans and other species.

In the method of the invention, the delta ¹⁴C values measured from a biomolecule is compared to a chart of historic delta ¹⁴C values—also referred to as a bomb-spike delta ¹⁴C chart. In a preferred embodiment, the bomb-spike chart is one of the many bomb-spike charts presented in FIG. 1. In a more preferred embodiment, the bomb-spike chart is chosen from FIG. 1A, 1B, 1C, 1D, and 1E.

The term biomolecule, for the purpose of this disclosure refers to one or more biomolecules. So, for example, a biomolecule may be a DNA molecule, a collection of DNA molecules, a chromosome, a cell, a whole tissue section, an organism (including an animal, a plant, or a virus). Where the biomolecule comprise more than one molecule type, the birth date is the average birth date of all the biomolecules being analyzed.

One embodiment of the invention is relates to a method for determining a birth date of a biomolecule comprising. In the method, a carbon containing biomolecule is provided. A delta ¹⁴C value is determined from the biomolecule. Then a birth date of the biomolecule is determine by comparing the delta ¹⁴C value of said DNA with a calibration delta ¹⁴C chart. Any of the charts in FIG. 1 may be used to determine a birth date. In particular, charts shown in FIG. 1A, 1B, 1C, 1D and 1E are preferred.

In one aspect of the method, the biomolecule is a whole tissue, such as, for example, a brain section, a liver section, a heart section and the like. In another aspect, the biomolecule is isolated from a tissue. In those aspects, the biomolecule can be an intracellular molecule such as DNA. The biomolecule may comprise a whole animal (e.g., small or unicellular animal), a plant or a virus. In another aspect, the biomolecule may be a purified cell population such as, for example, an isolated neuronal cell population, spleen cell population, liver cell population and the like. The cell population can be further purified by the use of known techniques such as FACS. The biomolecule may be a DNA purified from any of the tissues, cell populations, and organisms listed in this disclosure. In another aspect, the purified cell population may be further purified according to a secondary birth date sorting method before delta ¹⁴C determination. An example of such a method involves the use of FACS to sort cells according to histone acetylation levels, DNA oxidation levels, cellular lipofuschin levels, or a combination thereof. The delta ¹⁴C level of cells sorted by the secondary birth dating method can be measured. Alternatively, if it is just desired to determine a spread of birth dates, after determining the birth dates by the secondary birth dating method, the cells can be combined for a determination of an average birth date by delta ¹⁴C.

The delta ¹⁴C value, of any of the methods of this disclosure, may be measured by any known means such as, for example, scintillation counting. The preferred method for delta ¹⁴C measurement is by an accelerator mass spectrometer.

Another aspect of the invention relates to a method of determine the birth date of a biomolecule in a organism population. In the method, a sample of said biomolecule from an organism population is collected and purified away from other carbon containing molecules of the organism population. Then a delta ¹⁴C level is determined for the biomolecule. The delta ¹⁴C level is compared to a calibration delta ¹⁴C chart (also known as a bomb spike chart) to determine a birth date of the biomolecule.

In one aspect, the biomolecule may be a tooth enamel from an animal. While any animal may be used, the preferred animal is a mammal such as a human, a horse, a pig, a cow, a rabbit, a dog, a rat and a mouse. A birth date may be calculated by knowledge of when the enamel is generally formed in an animal. For example, in a horse, the enamel of the incisors are generally 6 years younger than the birth date of the animal. Thus, if the enamel shows an age of 10 years, the horse would be about 16 years old. This method can be generally applied to any animal where the enamel date relative to birth date is known.

Another aspect of the invention relates to a screening method for determining if a candidate agent have an effect on cell proliferation. In the method, a sample tissue is taken from the animal and tested for it's birth date using any of the methods of the invention. Then the animal is administered the' candidate agent. After administration, another sample tissue, similar in type and location is collected from the animal and the tissue's birth date is determined. The two birth dates are compared to determine if cell proliferation has occurred. If cell proliferation has occurred, the birth date of the tissue is expected to decrease (younger) representing new cell proliferation. Any tissue may be tested. Example of tissues include any tissue in this specification and at least, includes neuronal and CNS tissue, liver, spleen, heart, and pancreas.

Another aspect of the invention relates to a screening method for determining if a treatment has an effect on cell proliferation. In the method, a sample tissue is taken from the animal and tested for it's birth date using any of the methods of the invention. Then the animal is administered a treatment. After the treatment, another sample tissue, similar in type and location is collected from the animal and the tissue's birth date is determined. The two birth dates are compared to determine if cell proliferation has occurred.

Treatment may encompassed any treatment such as, for example, electroshock, trauma, an induced disorder, a surgical procedure, and the administration of an agent.

Another aspect of the invention is directed to a method for determining a birth date of a biomolecule. In the method, a biomolecule is provided. An isotope concentration in the biomolecule is determined. The isotope may be any isotope that exhibit uniform changes of concentration with time. Exemplarity isotopes include nitrogen and carbons. A birth date of the biomolecule may be determined by isotope concentration with a calibration isotope concentration chart.

In any of the method of the invention, the determination of the birth date of a biomolecule may be used to determine the birth date of a cell, a tissue, or an organism comprising the biomolecule. For example, DNA is usually produced during cell birth. The birth date of DNA will reflect the birth date of the cell, and the tissue where the DNA is collected.

### BRIEF DESCRIPTION OF THE DRAWINGS

FIG. 1 depicts (a) delta ¹⁴C values in atmospheric CO₂; (b) Northern and Southern hemisphere delta ¹⁴C values; (c) Northern hemisphere delta ¹⁴C values from 1958 to 1997; (d) bomb-spike curve from 1890; (e) bomb-spike cure from 1970 to present; (f) bomb-spike curve from 1985 showing an age determination for the cerebellum and cortex of a 19 year old horse; (g) bomb-spike curve for from 1985 showing an age determination for the cerebellum and cortex of a 19 year old and a 6 year old horse; (h) bomb-spike curve from 1985 showing an age determination for the blood of a 19 year old and a 6 year old horse; (i) bomb-spike curve for from 1985 showing an age determination for the enamel of a 19 and a 6 year old horse; (j) bomb-spike curve showing an age determination for the DNA or various tissues (LV is lateral ventricle) in one human; (k) bomb-spike curve showing an age determination the DNA or various tissues (OB is ofactory bulb) for a second human; (l) bomb-spike curve for two humans from 1985 showing an age determination for the enamel and showing a correlation between enamel birth date and human birth date.

FIG. 2 depicts (a, top panel) a gel showing the purity of DNA preparation from various tissues, where the DNA is stained by ethidium bromide (molecular size standard on the left lane), (a bottom panel) a gel showing, from left to right, molecular weight reference, isolated DNA, isolated DNA treated with Dnase, isolated DNA treated with RNase; (b) bar graph showing 8 preparations of human DNA with negligible protein contamination, the figure is divided into 8 sections on the X axis, each section comprise two bars, the taller bar on the right in each section shows DNA content, the low bar on the left (barely above zero) in each section denotes protein content. The protein content is always a negligible and a small percentage of the DNA content.

FIG. 3 depicts the use of Ficoll and Percoll gradients for the purification of cells for birth dating.

FIG. 4 depicts DAPI, and NeuN staining of cells and nuclei purified by a gradient.

FIG. 5 depicts nuclei purified for birth dating.

FIG. 6 depicts the result of FACS analysis of fresh pig and frozen human nuclei.

### DETAILED DESCRIPTION OF THE INVENTION

The invention is directed to methods of determining the age of a biomolecule by determining the delta ¹⁴C value of the biomolecule. A biomolecule is defined as any carbon containing molecule synthesized by a living organism. The biomolecule may comprise, at least, DNA, RNA, proteins, fatty acids, oils and other carbon containing compounds in an organism (living or nonliving). One preferred biomolecule is DNA because DNA is synthesized at the time of cell division, thus, a determination of DNA birth date is also a determination of the cell birth date. However, it may be useful to determine the age (birth date) other biomolecules in a cell. For example, it is possible to determine the time of preservation of a preserved tissue sample, a frozen vaccine, a historic blood sample and the like by measuring the delta ¹⁴C value.

As stated above, because DNA is synthesized at a time which is close to the birth of a cell, and because DNA is not synthesized in a nondividing cell, the measurement of the age of a DNA molecule will provide an accurate indication of the age of a cell. Other carbon containing biomolecules that, like DNA, are synthesized at a time close to the birth of a cell but not synthesized during the life of the cell may be used to determine the age of a cell.

The delta ¹⁴C value may be observed in a sample by measuring its radioactive decay. Unfortunately, radio-isotopes that have longer half lives, such as ¹⁴C(5730 years), are inefficiently detected by decays. Measuring even 0.1% of the ¹⁴C's in a sample requires uninterrupted counting for 8.3 years (0.1%×5730 years/ln(2)). The sensitivity and specificity of the radioisotope label are thus lost in the detection of decays.

In the late 1970's and throughout the ‘80’s a mass spectrometric method for directly detecting ¹⁴C and other long lived isotopes was developed in low-energy nuclear physics laboratories. At present, ¹⁴C dating can been accurately accomplished by the use of particle accelerators to obtain highly positively charged carbon atoms which were then separated by mass spectrometry and then directly or indirectly counted.

The method of AMS utilizes a spectrometer that consists of a source, an accelerator and various detectors. The AMS accelerates a beam of carbon ions to very high energies. At high energy the carbon ion beam can be manipulated using large magnets so that the various isotopes (¹²C and ¹⁴C) get directed towards different detectors.

Briefly, the DNA is converted to pure carbon in the laboratory. This prepared sample is placed in an evacuated chamber, where it is bombarded with positive cesium ions (Cs⁺). Carbon ion beams, a result of cesium bombardment, are then accelerated in an accelerator. As the ions emerge from the accelerator they are separated by magnetic and electrical fields according to their mass, and then counted by various detectors. The accelerated carbons are analyzed by an analysis filter. Data received from ¹⁴C analysis is given in the form of delta ¹⁴C values.

Delta ¹⁴C calculation is a value corrected for isotopic fractionation and radioactive decay. Atmospheric ¹⁴C content is expressed as delta ¹⁴C, which is the relative deviation of the measured ¹⁴C activity from the NIST (formerly US National Bureau of Standards) oxalic acid standard activity, after correction for isotopic mass fractionation and radioactive decay related to age (Stuiver and Polach, 1977: Stuiver, M., and Polach, H. A. (1997) Discussion: Reporting of 14C data. Radiocarbon 19:355-363).

Since AMS was initially developed for the difficult task of geochronology, in which the highest level of the isotope is a function of its natural production, the sensitivity of AMS stretches from parts per billion to parts per quadrillion. Several magnetic and electric sectors are needed to reduce ion counts to low enough rates that the ion identification techniques can operate.

The efficient throughput, described in the last paragraph, is possible only with graphitic samples. To facilitate this analysis, all biological samples are combusted to CO₂ in individual sealed tubes, and the CO₂ is reduced to graphite on an iron or cobalt catalyst in a second sealed tube also containing zinc metal and titanium hydride (Vogel 1992).

Since AMS came out of the ¹⁴C-dating community, the unit “Modern” is also introduced. This is the concentration of ¹⁴C that would be present in the quiescent atmosphere due only to cosmic radiation. Two anthropogenic effects have had profound effects on the atmospheric concentration of ¹⁴C in the past century. The burning of fossil fuels to power the industrial revolution increased the amount of ¹⁴C-free CO₂ in the atmosphere from the mid-1800's. The atmospheric testing of nuclear weapons then greatly increased the amount of ¹⁴C in the atmosphere, doubling the concentration of ¹⁴C by the year 1963. This huge excess of ¹⁴C has been drawn out of the atmosphere and into the oceans with an uptake half life of 15 years since the atmospheric test ban treaty was signed in 1964. For this reason, the current atmosphere has radiocarbon equivalent to 1.1 Modern.

AMS measurements are done to 3-5% precision as measured by the standard deviation of 3 or more measurements of the ¹⁴C concentration. AMS is one of the few methods for quantitating molecules precisely over this range. Radiocarbon dating is a much more stringent application of AMS, and an International Intercomparison has shown that AMS is more precise than liquid scintillation, and as accurate as CO₂ proportional counting (See, e.g., Scott 1990).

In a preferred embodiment of the invention, a date of a biomolecule synthesis (and hence a date of cell birth) may be determined by consulting a ¹⁴C value curve to predict the corresponding date. ¹⁴C curves have been provided in the Figures of this disclosure. Any delta ¹⁴C curve in this disclosure may be used to predict the age of a biomolecule. Naturally, curves with higher resolution are preferred and curves that are appropriate for the sample (e.g., northern hemisphere delta ¹⁴C curve or southern hemisphere delta ¹⁴C curve) may be used to improve the resolution of a date prediction. However, it should be noted that even the use of a low resolution curve or a curve for a different hemisphere would provide a degree of accuracy previously unattainable.

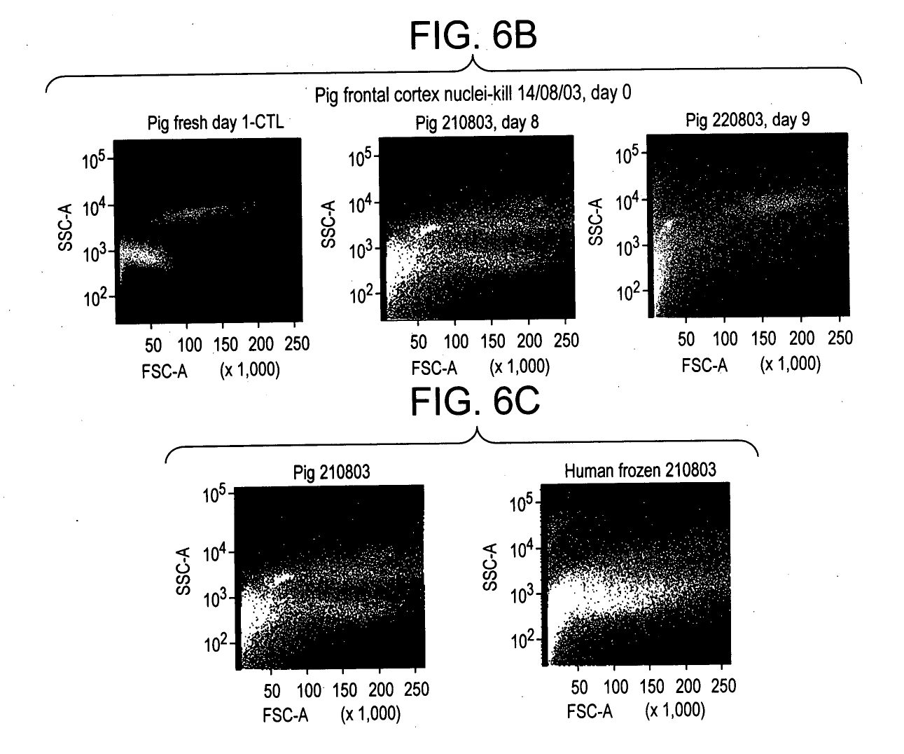

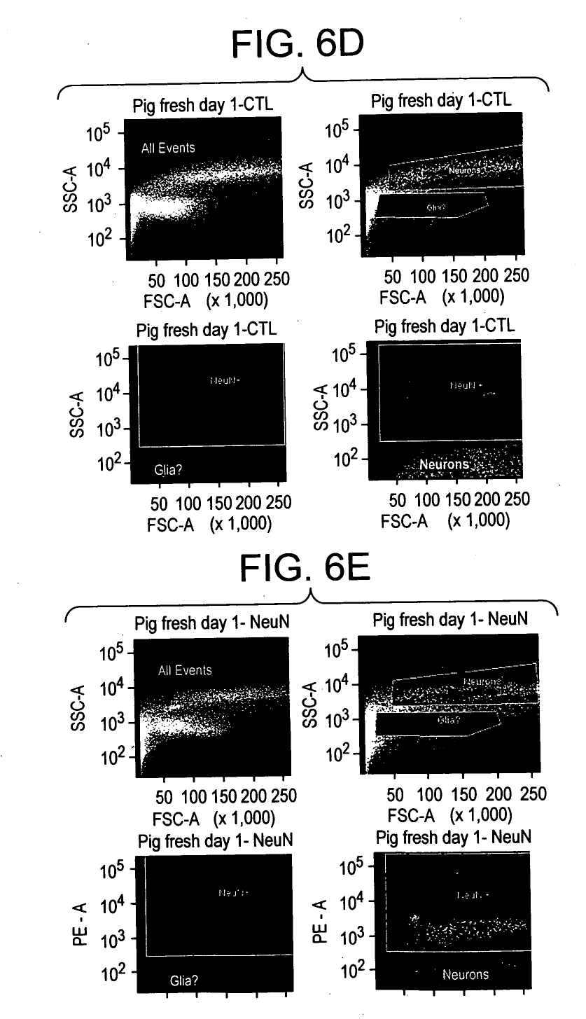

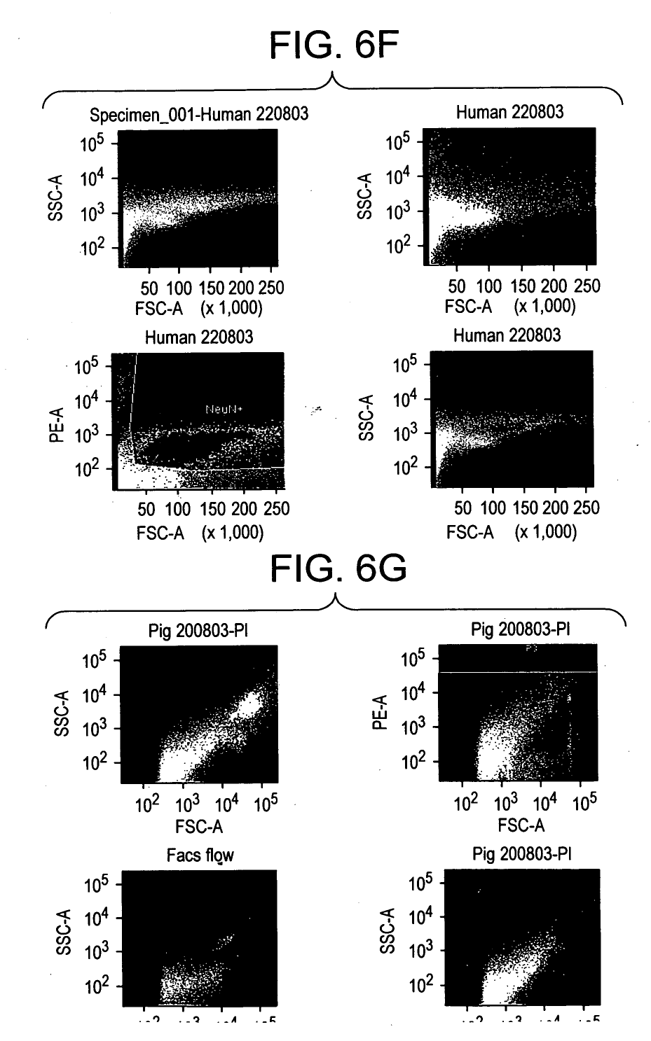

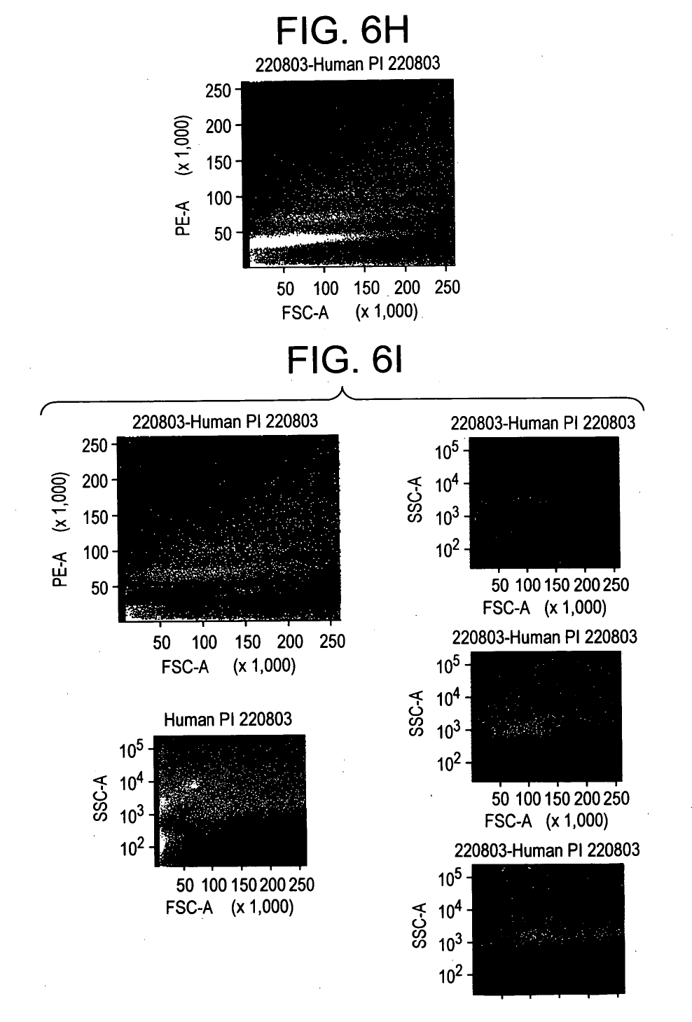

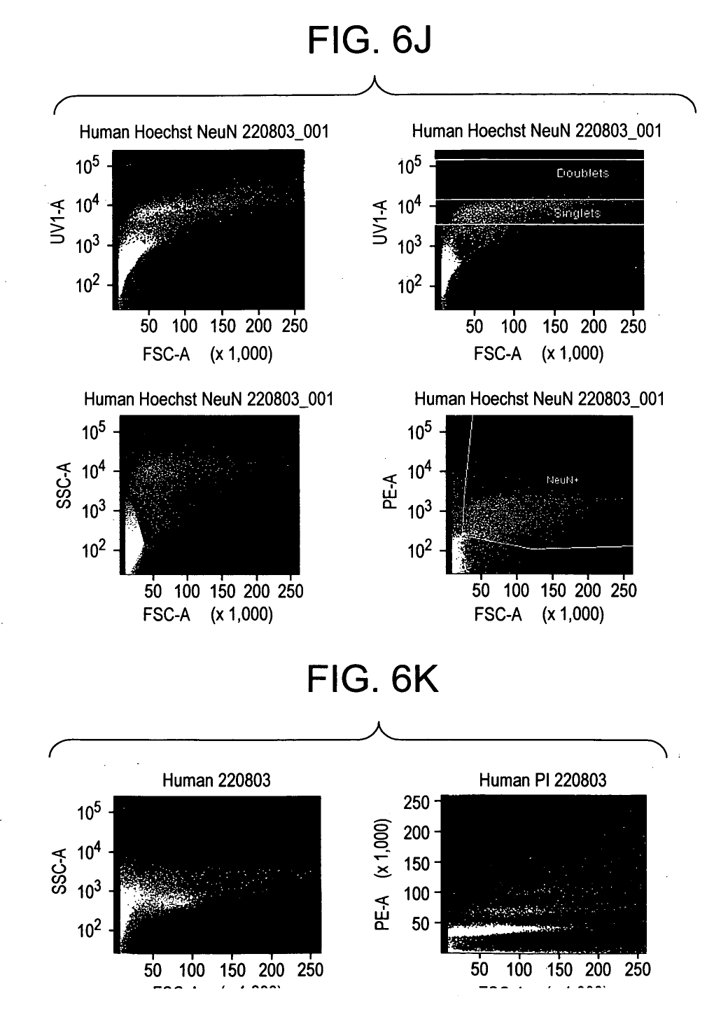

Delta ¹⁴C values have been collected world-wide from a variety of different regions—atmospheric (at varying levels), tree ring, coral and oceanic. The most comprehensive bomb-spike curve has been compiled by Ingeborg Levin (Levin, I., and B. Kromer, Twenty Years of Atmospheric 14CO2 observations at Schauinsland Station, Germany. Radiocarbon 39: 205 (1997), Levin, 1992; Levin et al., 1985) and covers multiple sampling regions in the Northern and Southern Hemisphere from 1890 until the present. In addition we have collected delta ¹⁴C curves from tree rings from Sweden (FIG. 6). This data importantly shows that ¹⁴C levels in sweden match that of atmospheric ¹⁴C levels published across the northern hemisphere. In a preferred embodiment, these readings are useful for determining a birth date in the Northern Hemisphere, such as, for example, in Europe or in Sweden. These data provide additional information lacking in the current literature for contemporary 14 C levels —most specifically in ventral Sweden.

The method of the invention may be applied to biomolecules, cells, tissues, and organs of any cell—including cells from any organism such as animals, plants, and viruses. The organism may be a mammal such as mice, cattle, sheep, goat, pigs, dogs, rats, rabbits, and primates (including human).

Other Applications Of The Birth Dating Methods Of The Invention:

The methods of the invention are suitable for determining the age of cells in the central nervous system (CNS). The development of the mammalian central nervous system (CNS) begins in the early stage of fetal development and continues until the post-natal period. The mature mammalian CNS is composed primarily of neuronal cells (neurons), and glial cells (astrocytes and oligodendrocytes).

The first step in neural development is cell birth, which is the precise temporal and spatial sequence in which stem cells and stem cell progeny (i.e daughter stem cells and progenitor cells) proliferate. Proliferating cells will give rise to neuroblasts, glioblasts and new stem cells.

The second step is a period of cell type differentiation and migration when undifferentiated progenitor cells differentiate into neuroblasts and gliolblasts which give rise to neurons and glial cells which migrate to their final positions. Cells which are derived from the neural tube give rise to neurons and glia of the CNS, while cells derived from the neural crest give rise to the cells of the peripheral nervous system (PNS).

The third step in development occurs when cells acquire specific phenotypic qualities, such as the expression of particular neurotransmitters. For example, at this time, neurons extend processes which synapse on their targets. Neurons are generated primarily during the fetal period, while oligodendrocytes and astrocytes are generated during the early post-natal period. By the late post-natal period, the CNS has its full complement of nerve cells.

The final step of CNS development is selective cell death, wherein the degeneration and death of specific cells, fibers and synaptic connections “fine-tune” the complex circuitry of the nervous system. This “fine-tuning” continues throughout the life of the host. Later in life, selective degeneration due to aging, infection and other unknown etiologies can lead to neurodegenerative diseases.

Unlike many other cells found in different tissues, the neurons of the adult mammalian CNS have no ability to enter the mitotic cycle and generate new nerve cells. While it is believed that there is a limited and slow turnover of astrocytes (Korr et al., J. Comp. Neurol., 150:169, 1971) and that progenitors for oligodendrocytes are present (Wolsqijk and Noble, Development, 105:386, 1989), the generation of new neurons does not normally occur. Therefore, neurogenesis (the generation of new neurons) is mostly complete early in the postnatal period.

Because DNA synthesis is completed at the time of cell birth and cell birth occurs at an early stage in life, the DNA in the CNS can serve as a reliable indicator of the age of the cells. Furthermore, the methods of the invention will allow for a more careful examination of cell division in the CNS. The methods of the invention are useful to determine the birth date, and hence the mitotic activity of adult neuronal cells. This technique is especially important given the recent findings in the stem cell field that show that neurogenesis continues into adulthood in mammals. Currently, there are no other method available to determine the birth date (including retrospective dating) or mitotic activity, which are not detrimental to the animal (including human) being studied.

The birth dating method of the invention may be used to study disorders. One example of a disorder is CNS disorders. Aberrations in normal neurogenesis have been linked to several neurological conditions. Stress has been shown to suppress neurogenesis (Gould, E., et al., (1998) Proc. Natl. Acad. Sci. USA 95:3168-3171), and a loss of neurons in the prefrontal cortex and hippocampus has been observed in depressed and anxious patients. Specific regions of the brain have been found to be smaller in chronically depressed patients than in their non-depressed counterparts (Czeh, B., et al., (2001) Proc. Natl. Acad. Sci. USA 98(22): 12796-12801.). Hippocampal neurogenesis has been shown to be required for the behavioral effects of antidepressants (Santarelli, L., et al., (2003) Science 301(8):805-809).

Disregulation of neurogenesis has also been linked to neurodegerative diseases, such as Parkinson's disease and Alzheimer's disease (Barzilai, A., and Melamed, E. (2003) Trends Mol. Med. 9(3):126-132; Tatebayashi, Y., et al., (2003) Acta Neuropathol. 105(3):225-232). There is accumulating evidence suggesting that various brain insults increase neurogenesis in the adult mammalian brain. Studies on dentate gyrus neurogenesis in adult rodent epilepsy models indicate that seizure-induced neurogenesis involves aberrant neuroblast migration and integration that may contribute to persistent hippocampal hyperexcitability; and that sub ventricular zone neurogenesis increases following stroke (for review see Parent, J. M. (2003) Neuroscientist 9(4):261-272). Accordingly, the study of cell turnover in the normal, diseased and injured brain would be of great interest to the scientist and clinician alike.

In one embodiment, the invention is directed to a method to determine the presence of neurogenesis in an organism. The organism may be any living organism including animals, plants, virus. The animal may be any mammal such as humans, horses, pigs, cows, rats, mice and the like. The methods of the invention may be applied to the tissues in said animal to determine an average birth date. The average birth date would allow the determination for the presence or absence of neurogenesis. Neurogenesis may result as part of normal development, as part of a neurological disease including neurodegenerative diseases, as part of a response to injury, or as part of a response to a drug administered to the animal. The methods of the invention may be used to study any condition and disorders where cell or biomolecule turnover is of interest. Furthermore, if the disorders are treated, for example, by inducing cell division or differentiation or by infusion of new cells, this process may be monitored by the methods of the invention.

Another important use for the methods of the invention is in the area of drug development and treatment development. A need exists for drug screening purposes and for the study of CNS function, dysfunction, and development. The mature human nervous system is composed of billions of cells that are generated during development from a small number of precursors located in the neural tube. Due to the complexity of the mammalian CNS, the study of CNS developmental pathways, as well as alterations that occur in adult mammalian CNS due to dysfunction, has been difficult. The methods of the invention may be used, for example, to monitor cell division in a CNS cell after treatment of a patient with a candidate drug.

One embodiment of the invention is a method for determining the birth date of a cell or a cell population. A birth date is defined as the date of the last cell division that give rise to the cell in question. Where biomolecules from multiple cells are pooled for analysis, it is understood that birth date refers to the average birth date of all the cells that contributed to the biomolecule sample. A birth date may be expressed as a year and a month. The accuracy of a birth date determination may be +/−5 years or less. In a preferred embodiment, the accuracy of birth dating is +/−3 years or less, such as, for example, +/−2 years or less or +/−1 year or less. In a most preferred embodiment, the accuracy of the birth dating is +/−6 months.

In one method of the invention, birth dating is determined by isolating a target cell population. The cell population may be any cell population in an organism. In a preferred embodiment, the cell population is a population that exhibits a low rate of cell division in an animal. While the method of the invention is not limited to cells with low cell division activity, the determination of a cell type with a high cell division rate, for example, blood cells is less useful because most blood cell have a birth date of less than one year. An example of a cell population with low cell division activity is neurons.

The first step in the study of diseases may involve the isolation of a cell population from an organism. Cell populations may be isolated by simple dissection. Alternatively, the cells may be dissociated from a tissue sample. The dissociated cells may be further sorted, or further purified, using known techniques such as fluorescent antibodies against receptors or cell-type specific protein and the use of fluorescence activated cell sorter. Other methods of cell purification include the use of density gradients such as, for example, a percoll gradient. It should be noted that the methods of the invention are not limited to require live cells. Thus, the range of cell purification techniques are not limited to those that preserve the viability of cells.

After isolation of a cell population, the DNA from the cell population is isolated. Part of the DNA isolation involves purifying the DNA away from all organic molecules (all non DNA carbon sources) from the cell population. Other organic molecules include RNA, proteins, fatty acids, membranes and the like. RNA may be removed from DNA using standard techniques such as, for example, RNase digestion.

A delta ¹⁴C value may be determined from the DNA. While many method of delta ¹⁴C are available, including, for example, liquid scintillation counting, the method of AMS is preferred.

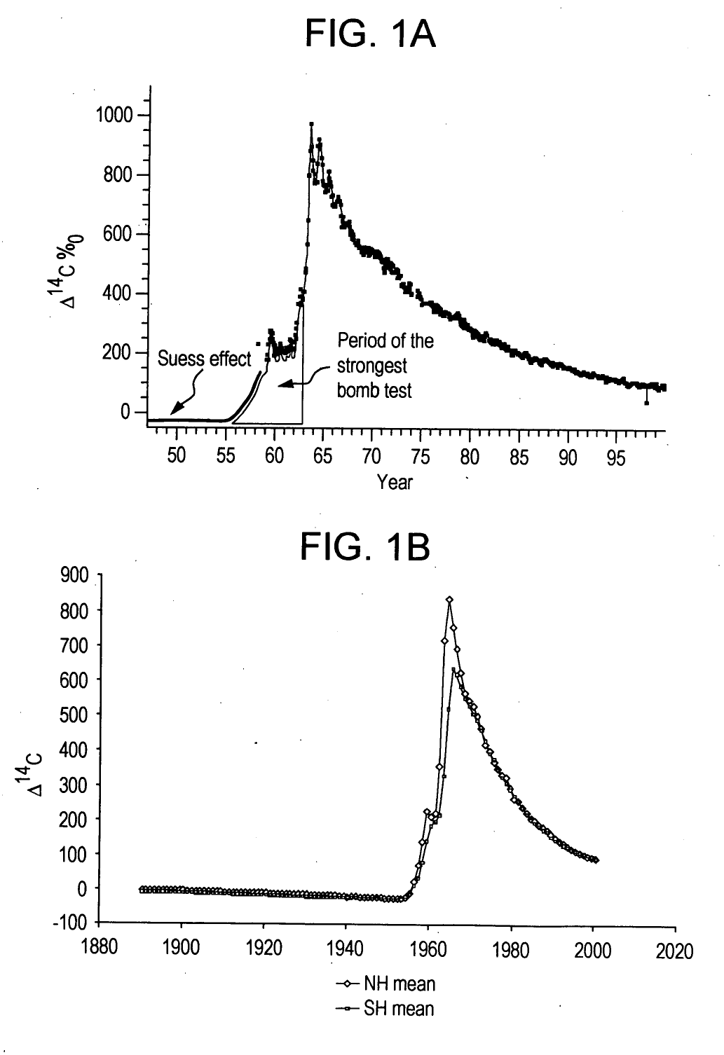

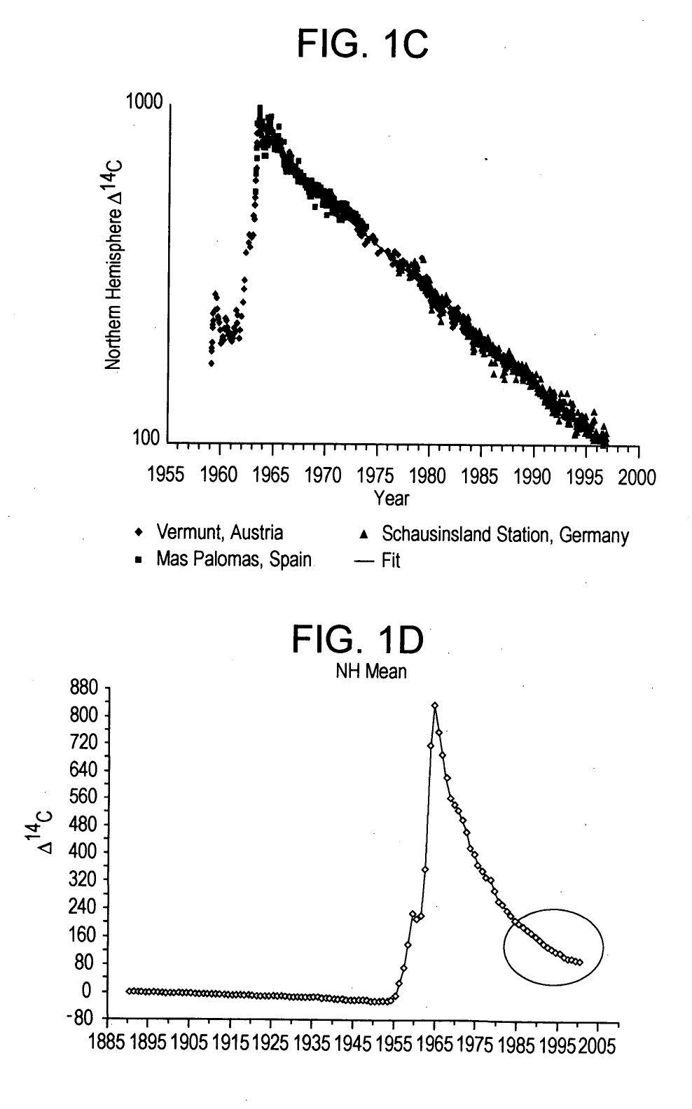

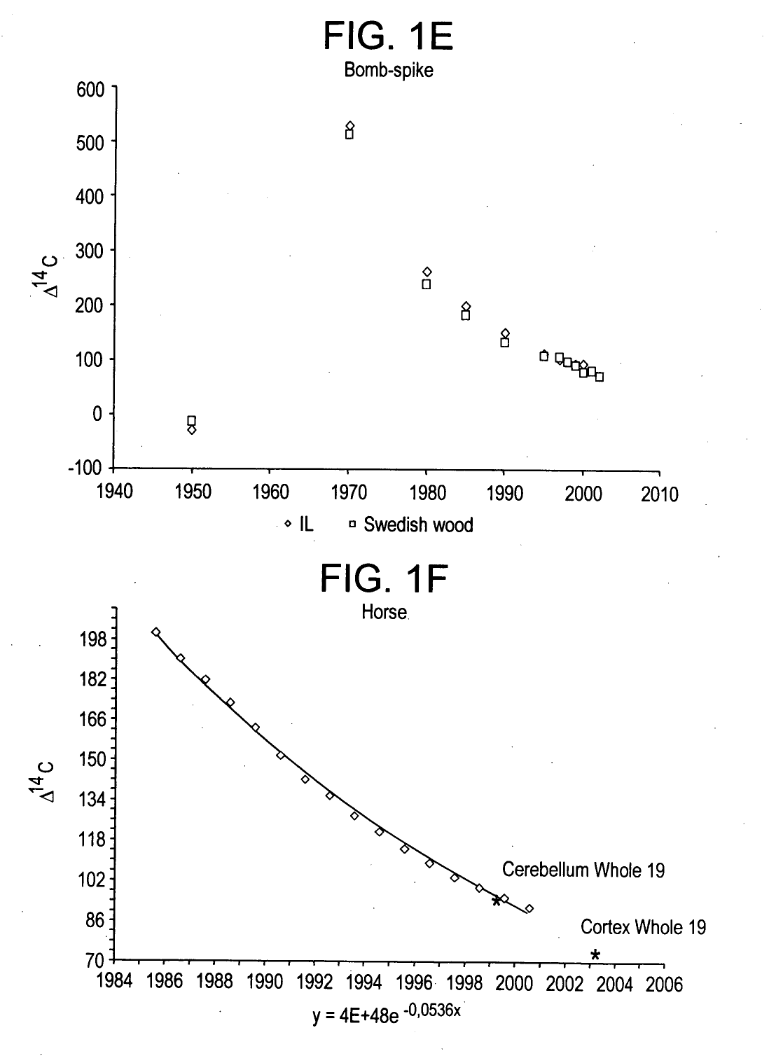

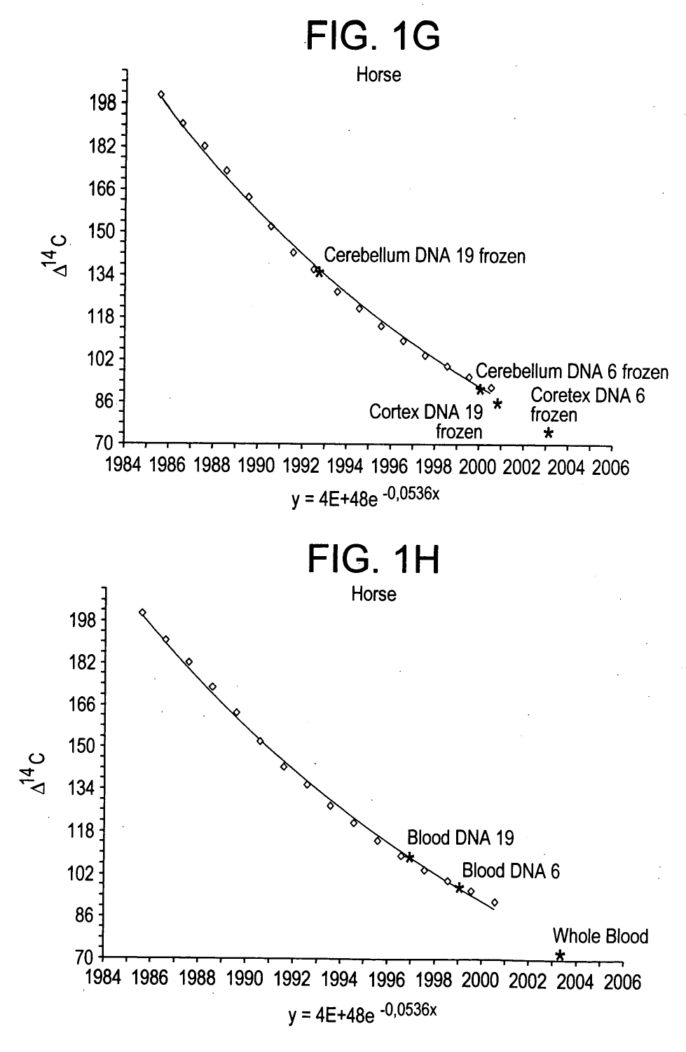

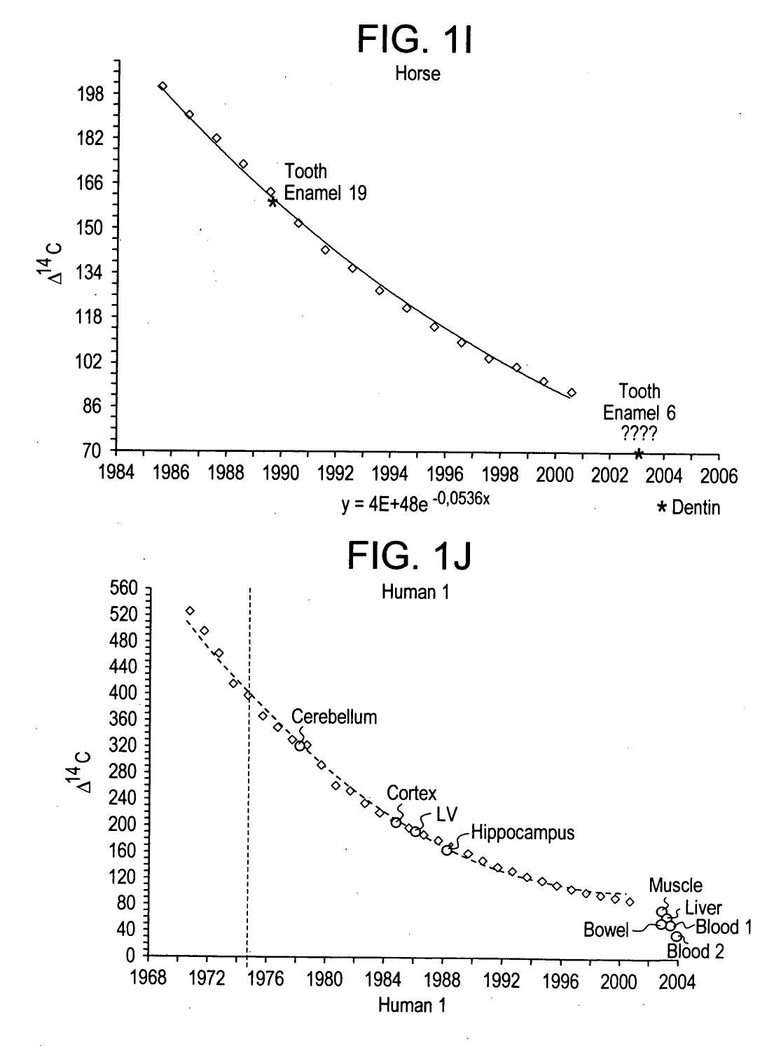

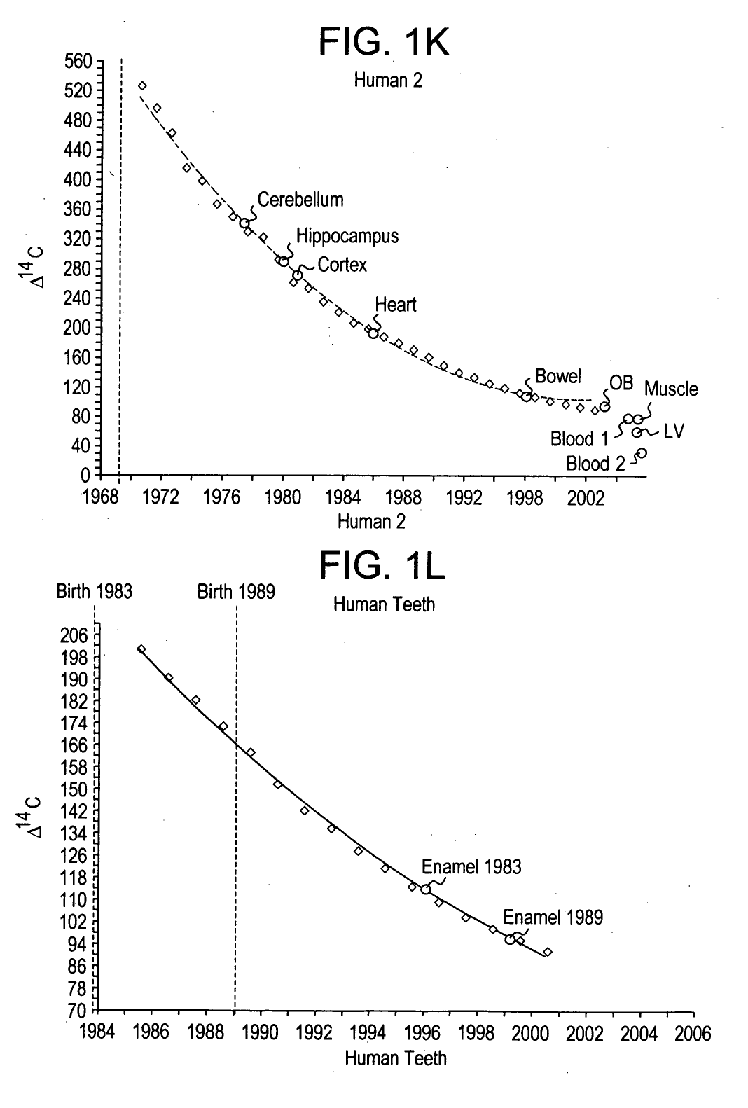

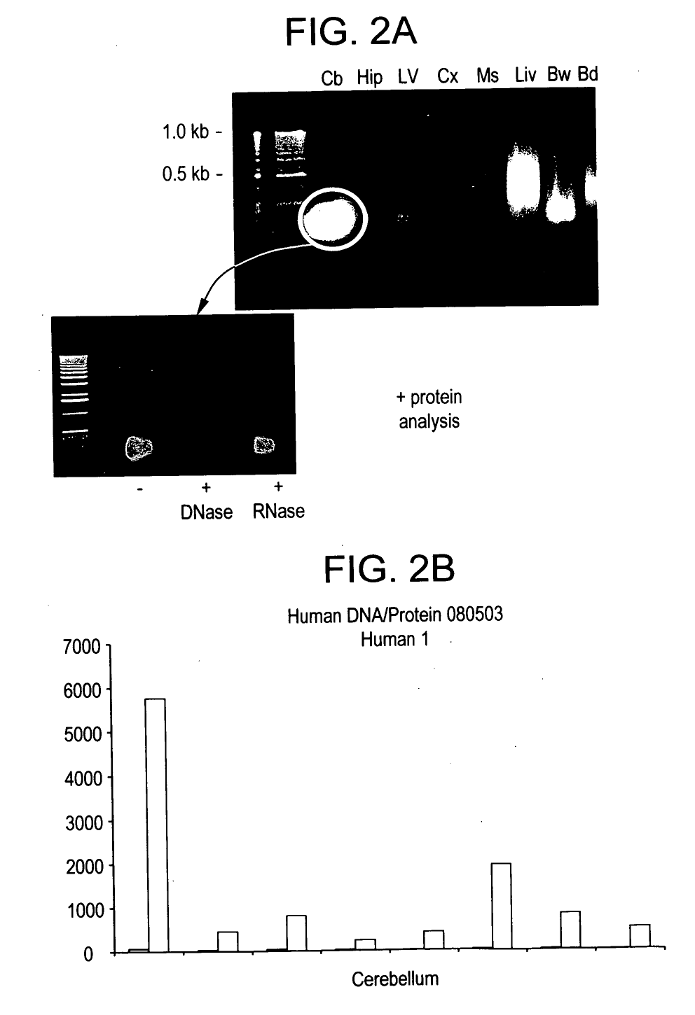

The synthesis date of the DNA may be determined by comparing the delta ¹⁴C value of the DNA to a chart of FIG. 1. For example, the delta ¹⁴C value may be compared against FIG. 1A to determine it's birth date. Alternatively, for better resolution, if the origin of the source of DNA is known, a more detailed chart, such as the northern hemisphere specific or southern hemisphere specific chart of FIG. 1B may be counsulted. Similarly, the age of DNA sample collected near Austria, Spain or Germany may be determined by consulting FIG. 1C. The birth date of the DNA sample is thus determined.

Since DNA synthesis begins at about the same time as cell birth, the birth date of the cell population may be determined from the age of the DNA. Furthermore, if the cell type is known and the life cycle of the cell type in an animal is known, the age of the animal may be determined. For example, the perkingi cells and cerebellum cells have birth dates around the time of birth, liver cells turn divide approximately every three months and many blood cells undergo division weekly.

The method of the invention may be use to calculate the birth date of any DNA, tooth enamel, cell, or organism with any birth date. In one embodiment, the birth date of the organism is 1963 or later. In another embodiment, the method of the invention is used to calculate a birth date that is after 1964 or later. In a more preferred embodiment, the methods of the invention is used to determine the birth dates from 1965 or later.

Another embodiment of the invention is directed to a method for determining the birth date of a teeth enamel. In the method, a sample of tooth enamel is collected from an animal and purified away from other carbon containing molecules. Other carbon containing molecules include the other parts of the teeth such as the dentin and the pulp. A delta ¹⁴C value is determined from the tooth enamel. In a preferred embodiment, the delta ¹⁴C value is determined by AMS. The age or birth date of the enamel may be determined by comparing the delta ¹⁴C level of the teen enamel with a chart from FIG. 1. From the birth date of the tooth enamel, the age of an animal may be calculated.

Screening Therapeutics

The method of the invention may be used to screen agents for the treatment of disorders. The term “agent” refers to something that may influence a biological condition. Often the term will be synonymous with “stimulus” or “stimuli” or “manipulation.” Agents may be materials, radiation (including all manner of electromagnetic and particle radiation), forces (including mechanical, electrical, magnetic, and nuclear), fields, and the like. Examples of materials that may be used as agents include organic and inorganic chemical compounds, biological materials such as nucleic acids, carbohydrates, proteins and peptides, lipids, and mixtures thereof. Other specific examples of agents include non-ambient temperature, non-ambient pressure, acoustic energy, electromagnetic radiation of all frequencies, the lack of a particular material (e.g., the lack of oxygen as in ischemia), etc. The term agent also refers to growth factors involved in neural development. These growth factors includes, but are not restricted to, NGF, NT-3, NT4/5, IGF-1, estrogen, PDGF, bFGF, IGF-1 and 2, NT-3, CNTF, retinoic acid, IL-6, and LIF.

In Parkinson's disease, a loss of 60% of substantia nigra cells results in the manifestations of clinical symptoms including bradykinesia and tremors. Current therapies are directed at replacing the deficient neurotransmitter, dopamine, or maintaining its presence by blocking its metabolism. By injection of various candidate agents (potential therapeutics), including neuronal cells, a treatment for Parkinson's disease may be explored.

For example, a candidate agent, dopaminergic cells (neural stem cells, primary cells from the basal ganglia, limbic system, substantia nigra, hypothalamus, the medulla cortex or other cells lines of neural or adrenal origin (such as PC 12)) or dopamine may be administered to a patient suffering from Parkinson's disease. By monitoring average birth date in the substantia nigra before and after the administration, the researcher can determine if neurogenesis has occurred in response to the administration. That is, if neurogenesis has occurred, the average age of the cells in the substantia nigra should drop, to reflect a younger population of cells. The method of the invention, by directly measuring cell division as evident by novel DNA synthesis, is also suitable for determining if an agent can elicit neurogenesis through an indirect, pleotrophic, effect (e.g., by secondary messengers etc).

This method can be applied to multiple disease paradigms, for example, Alzheimer's disease. Briefly, the average birth date of the basal forebrain is determined. Then a candidate agent is administered to the patient. After administration, the average age of the basal forebrain is determined again to determine if the agent has induced neurogenesis in the basal forebrain.

While two specific examples, Parkinson's disease and Alzheimer's disease have been discussed above, the method of the invention is suitable for the screen of any agent for the treatment of any disease in a patient which is in some way associated with cell division. The cells assayed by the methods of the invention may be any type of cell.

Neuronal cells may be analyzed after their isolation from a tissue. As shown in the Example section, neuronal cells may be purified or enriched by flourescent activated cell sorting or other cell sorting techniques. After a cell population is enriched for neuronal cells, the birth date of the cells may be determined by the methods of the invention.

One advantage of the method of the invention is that it does not rely on live cells. In fact, the methods of the invention is equally applicable to dead cells. Because of this, the methods of the invention may be applied to dead tissue for scientific or forensic purposes. Another advantage of the methods of the invention is that it is applicable to all cells, regardless of their origin, as long as the cells have a biomolecule that can be analyzed. So for example, vegetable cells may be analyzed with the same accuracy as animal cells. Furthermore, more than one type of biomolecules may be analyzed by isolating the molecule and determining a delta ¹⁴C level. In any of the methods of the invention, the biomolecule may be a whole cell or a whole tissue. The delta ¹⁴C level of whole cells can be determined without purification of cellular components.

Another technique that can be used to further refine the age distribution of cells or nuclei includes dating cells (including neurons) based on their level of a cell age indicator.

One cell age indicator is cell lipofuscin. All cells accumulate a product called lipofuscin with time. The exact molecular composition of this pigment is not fully characterized. However, lipofuschin has a yellow and green autofluorescecent property. This property can be induced using light of 400 to 600 nm to excite the lipofuschin and measuring autofluorescence at 400-640. We can take advantage of the fluorescent properties of lipofuscin to by flow cytomtery isolate subpopulations that have varying levels of lipofuscin, and determine the age of subpopulations with the above described ¹⁴C method of cells with varying levels of lipofuscin.

Another cell age indicator is histone acetylation. Histone acetylation may be measured by many methods. Two of these methods involves directly adding a florescently tagged antibody to neuronal nuclei that has been extracted and purified from a cell (discussed in another section of this disclosure). The second method involves extracting histones from from NeuN+sorted neurons. The histones extracted are then labeled with an anti-histone fluorescent-conjugated antibody (such as Alexa Fluor 546, Zenon One kit from Molecular Probes). Briefly, histone extraction involves extracting histones with 0.2M H₂SO₄ and then precipitating with four volumes of ethanol, and redissolved in 0.9M acetic acid containing 15% sucrose (Serra et al., 1986). Labelled histones are then sorted for acetylation level by running the nuclei or histone population through a FACS sorter (fluorescence-activated cell sorter). Highly acetylated, and thus old cells, will fluoresce much more than younger, less acetylated cells, giving additional information as to the proportion of young and old cells in a given population. Other methods for purification of histones and determining histone acetylation levels are disclosed in U.S. Pat. No. 6,068,987.

A third indicator of cell age is DNA oxidation. DNA oxidation is assessed by looking at oxo8dg levels. Oxo8dg levels have been shown to increase in an age-related manner in all tissues of rodents (Hamilton et al., 2001). Oxo8dg can be directly detected by avidin and its analogues, and represents an additional method for age sorting nuclei. Again, analysis would be by way of FACS sorting of fluorescent-labelled avidin bound oxo8dg (Struthers et al., 1998).

In each case, cells can be sorted (by FACS if needed) by the cell age indicator into different subpopulations. Each subpopulations may be subjected to a ¹⁴C analysis method of the invention to determine its birth date. In this way, the methods of the invention may be used to obtain a more refined picture of the turn over of cells.

### EXAMPLES

### Example 1

For AMS analysis of extracted DNA to be successful and accurate, three aspects are preferred: 1) there needs to be a high yield of DNA; 2) the DNA needs to be as pure as possible; and 3) radioactive or carbon contamination should be eliminated or minimized. Mass spectrometric analysis of human cerebellar DNA has revealed that between 23 and 30% of the total mass of DNA is comprised of carbon. To optimize measurement accuracy, approximately 200 μg or more DNA was extracted from each sample for measurement.

Different DNA extraction protocols were investigated for their purity and yield of DNA. Two techniques were established which give either a high yield of DNA (phenol extraction method from Sambrook et al., (1989) Molecular Cloning—A Laboratory Manual, 2nd Edition, Cold Spring Harbor Laboratory Press) or a highly purified DNA sample (Dingley, K. H., et al., (2003) DNA isolation and sample preparation for quantitation of adduct levels by Accelerator Mass Spectrometry. Mol. Tox. Prot.; in press). These methods were successfully used in the disclosed experiments to extract DNA from mouse, horse, pig, and human material. In all cases, experiments were conducted with the utmost care in a sterile and radioactive-free zone.

It is noted that any DNA extraction procedure will work for the method of the invention so long as the final DNA extraced is clean of all carbon contining containments. One method of DNA extraction is provided as an example. Whole tissue can be homogenized in a tissue homogenizer and subjected to RNase treatment. After RNase treatment, the DNA is extracted with Phenol/chloroform/isoamyl alcohol to remove non-nucleic acid components. The DNA of the aqueous phase of the extracted preparation may be precipitated by ethanol and 0.3 molar (final concentration) sodium acetate. The precipitated pellet may be washed with 70% ethanol and allowed to dry. The DNA is then resuspended in clean H₂O and an aliquot can be taken for DNA and protein and protease contaiminating. The dried DNA samples are sent for AMS for ¹⁴C levels.

Various types of tissue were collected, such as brain tissue (cerebellum, cortex, hippocampus, and, in particular, dentate gyrus, lateral ventricle, and olfactory bulb) and other tissues, including muscle, liver, bowel, heart, and blood. Teeth were collected from horses and humans. Whole blood was collected, representing the newest source of ¹⁴C in the body. Tooth enamel was also collected to represent the oldest source of ¹⁴C in the body. Once enamel has been laid down it is not renewed or modified, and therefore its ¹⁴C content will reflect the age of the animal at the time of tooth formation. Both of these samples were used as internal controls for each subject.

Experiments on whole tissue DNA extractions were conducted with horse and human tissues, and showed excellent results. Analysis was performed for whole tissue, DNA from specific brain and body regions, and tooth enamel. The delta ¹⁴C values were compared to one of the bomb-spike curves in FIG. 1A, 1B, 1C, 1D or 1E. As one would expect with whole tissue, the ¹⁴C levels were consistent with contemporary synthesis (FIGS. 1F and 1G). We would expect this since whole tissue contains many components that exhibit rapid turnover, such as proteins. Many cells that are known to rapidly turn over, such as blood cells, show a young birth date (FIGS. 1J and 1K). The DNA of cerebellar tissue was determined to be several years younger than the DNA of cortical tissue in both horse and human (FIGS. 1G, 1J and 1K). Again this result is in line with what would be expected since the cerebellum is a much more neuronally dense structure than the cortex, and the cortex contains a higher percentage of more proliferative (and thus younger) glial cells.

DNA was also extracted from various regions in the horse and human brain and body, including the cerebellum (a site expected to have no neurogenesis), cortex (a proposed site of adult neurogenesis), the lateral ventricle and the hippocampus (sites shown to be highly neurogenic), and muscle, liver, bowel, and blood (sites shown to have high rates of cell turnover). DNA from the cerebellum was determined to be very old (FIGS. 1G, 1J and 1K)—almost as old as the subject. Cortical DNA was determined to be about 6 years younger, as would be expected given the abovementioned differences in neuronal composition. The lateral ventricle, as expected given its neurogenic properties, was determined to be younger than cortex (FIG. 1J).

As expected, the regions with high cell turnover rates, i.e. muscle, liver, bowel and blood DNA, were showed ¹⁴C levels consistent with contemporary synthesis (FIGS. 1J and 1K). The sum of these results demonstrated that ¹⁴C measurements could successfully be used to date chromosomal DNA as well as whole tissue and structure containing carbon such as enamel (FIG. 1I), tissue containing said DNA, organs containing the tissue, and the animal containing the organs. That is, from a determination of the birth date of DNA, the age of the cell, the tissue, the organ, and the organism may be calculated. The technique is not restricted to ¹⁴C analysis, any other compound which demonstrates properties similar to ¹⁴C may be used.

Enamel from horse and human also show good correlation with age (FIGS. 1I and 1L). The tooth type dated for the horse is born around 5 years old and the age of the 6 year old horse is near contemporary. As expected and then enamel from the same tooth type in the 19 year old horse is about 14 years old—as expected.

### Example 2

Additional experiments were performed to further study the differences in ¹⁴C values between different cell types across a variety of different regions, and thus determine directly the level of neurogenesis. For these experiments, neurons were separated from glial cells and neuronal DNA was collected. This was done by sorting neuronal nuclei from the total nuclei population. Nuclei can be sorted by any nuclei extraction protocol. For this example, nuclei were isolated from whole tissue by mechanically homogenizing the tissue, lysing the cells in a lysis solution containing dithiothreitol and Triton X-100, and purifying the nuclei through a series of sucrose gradients. As small aliquot of nuclei were taken out for analysis, some of them were labelled with trypan blue and were quantitated by light microscopic analysis using phase contrast. Nuclei were then labeled with DAPI (a nuclei stain) and incubated with the neuronal antibody NeuN (neuronal nuclear protein) conjugated to fluorescent marker Alexa Fluor 546 (Zenon One Mouse IgG labeling kit; Molecular Probes).

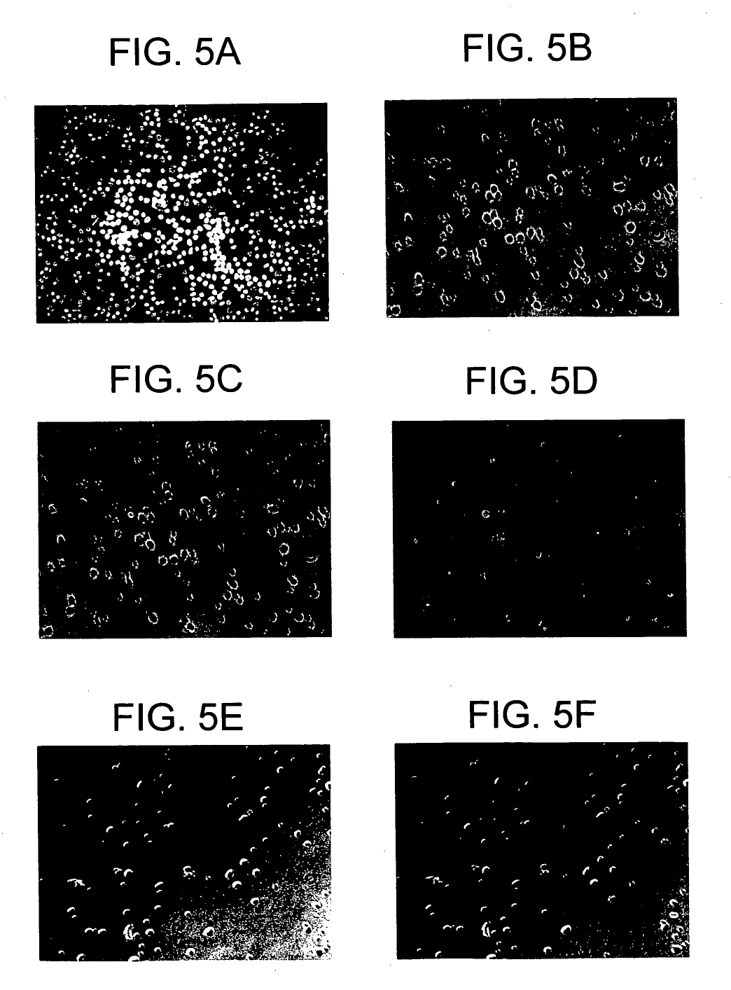

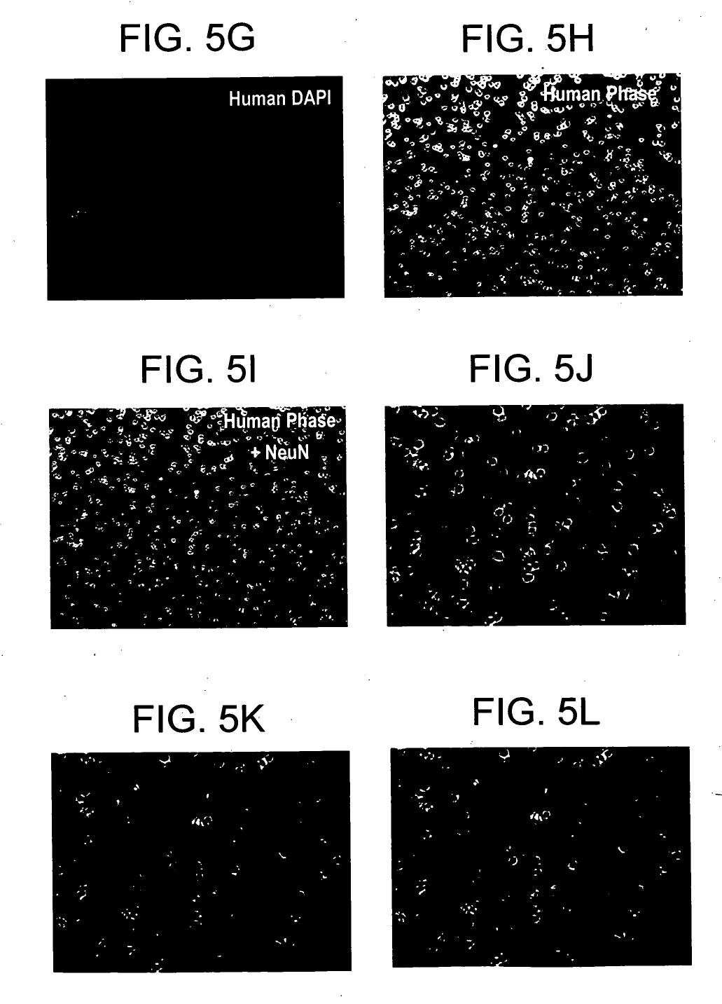

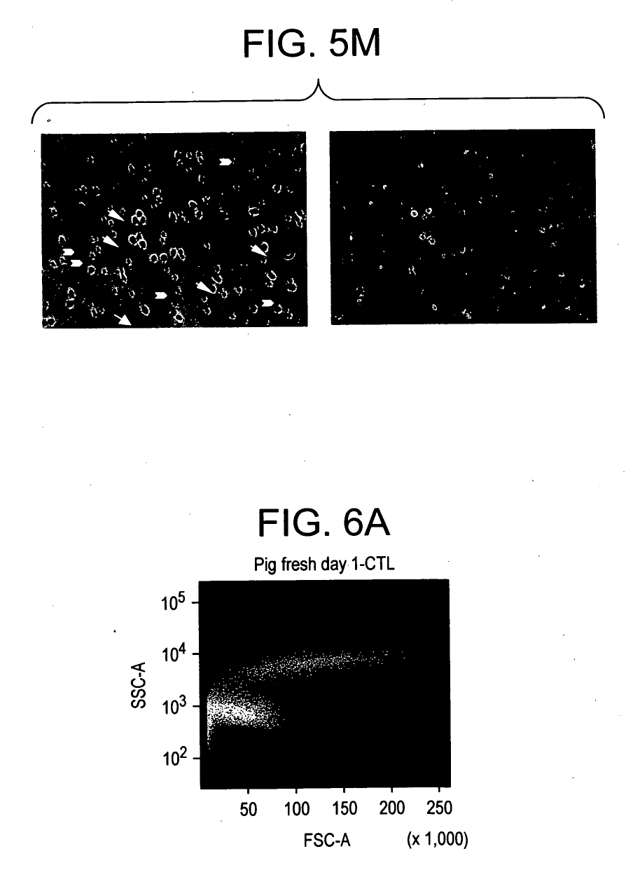

Fluorescently labelled nuclei were visualized using a fluorescent microscope, and the presence of NeuN-labeled nuclei confirmed. See FIG. 5a, showing DAPI stain of nuclei. Neuronal nuclei could easily be be distinguished from glial nuclei when stained with DAPI or Hoechst 33342 (Spalding et al. (2002)). Microscope analysis of DAPI labeled nuclei and NeuN positive nuclei indicated the extent of neuronal labeling. Preliminary experiments with fresh pig cortical nuclei and frozen human nuclei showed extensive and robust NeuN labeling of neuronal nuclei. See FIGS. 5B and 5E showing phase contrast view of pig cells; FIG. 5C, and 5D and SF showing presence of NeuN positive nuclei (shown in pink). Similar results are seen for human nuclei preparations. See FIGS. 5G and 5J, showing nuclei prep stained with DAPI. See, FIGS. 5H and 5K, showing phase contrast view of human nuclei prep. See, FIGS. 51 and 5L, showing the presence of neuronal cells that stain positive with NeuN stain (pink). The presence of neuronal cells (thin arrows) and glial cells (thick arrows) are shown in FIG. 5M where neuronal or glial morphlogical analysis of a phase contrast lineage (left box) was confirmed with NeuN labeling (right box).

The cell and nuclei purification described above was performed on fresh pig cells and frozen human cells. The positive results and the successful isolation of neuronal nuclei indicate that show that it is possible to analyze a variety of healthy and diseased brain material stored as frozen material in pathology and forensic centers around the world.

Whilst a small aliquot of the extracted nuclei were used for microscopic analysis, the bulk of the material was prepared for analysis and sorting using fluorescence-activated cell sorting (FACS). Nuclei were labeled with NeuN, as described above, as well as with a DNA stain, such as propidium iodide (PI). PI labeling allowed single nuclei to be sorted from doublets, triplets, etc. (FIG. 6H to 6K). This ensured that sorting was performed for a neuron-specific population, and that glial nuclei were not adhered to neuronal (NeuN+) nuclei. Nuclei were sorted after extraction from fresh pig brain, aged but not frozen pig brain, and frozen human brain. The results indicated that sorting could be used with near-perfect accuracy for a neuron-specific population using PI and NeuN+gating (FIG. 6A-6G).

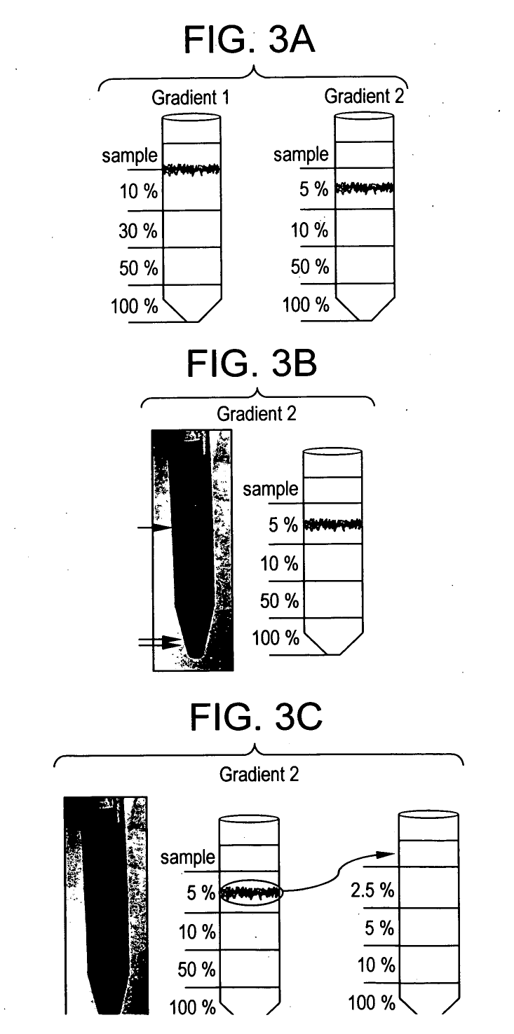

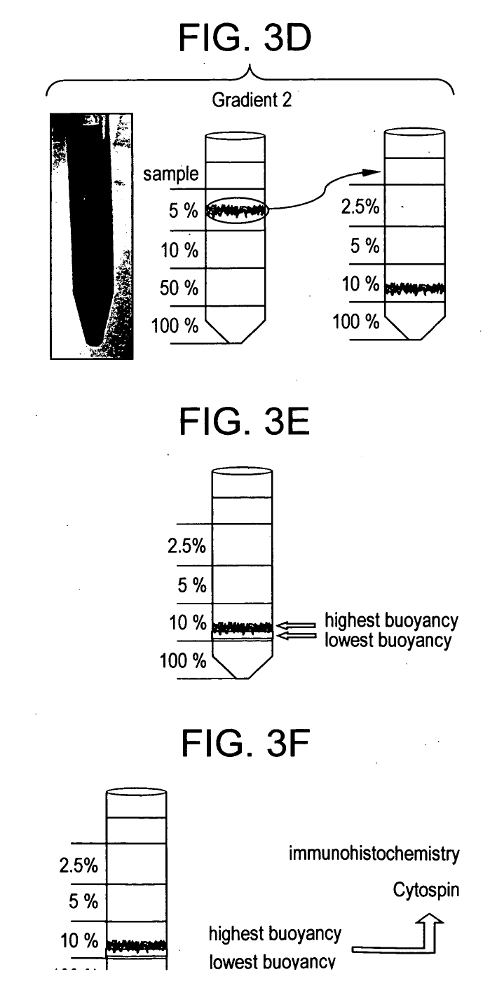

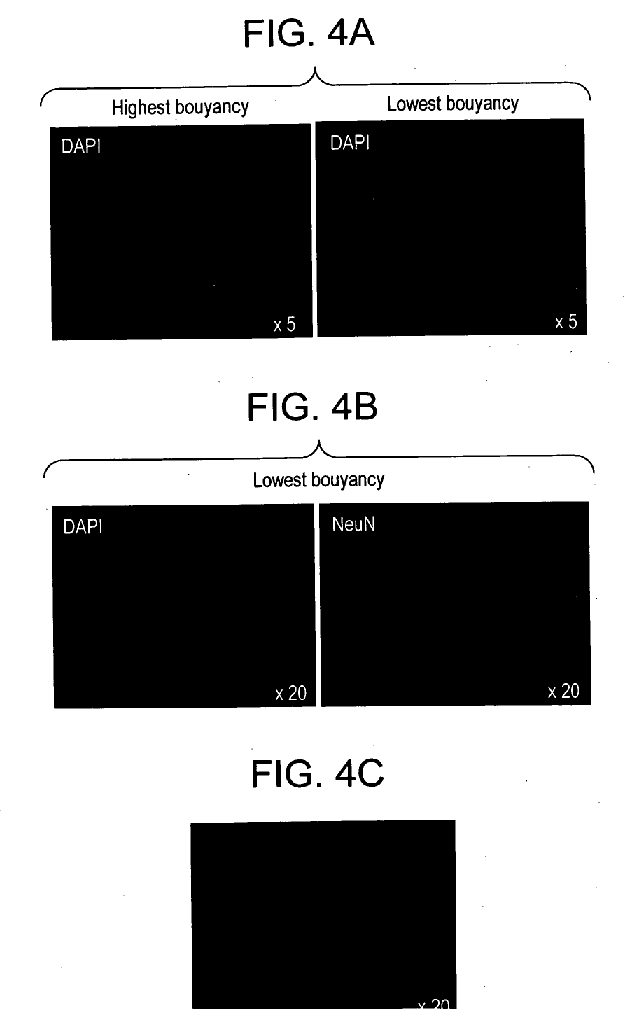

Whole cells were also separated on Percoll gradients (FIGS. 3A and 3F). In a Percoll gradient, two distinct populations of debris and cells of high and low buoyancy cells, can be distinguished. FIG. 4A shows DAPI nuclear staining of the cells of both fractions. The data indicates that a majority of cells are present in the lowest buoyancy fraction. The lowest buoyancy cells were further analyzed by nuclear (DAPI—blue in the figure) staining and staining specific for neuronal cells (NeuN—pink in the figure). FIG. 4C is a composite of the two figures in 4B.

Once a neuron-specific population of nuclei was collected, the DNA was extracted, cleaned, dried and resuspended in H₂O. The DNA was then quantitated using a spectrophotometer, and purity was assessed. Agarose gel analysis was performed with and without DNase and RNase pre-incubation to determine the presence of RNA. A micro-Bradford assay was performed to determine the presence of protein. HPLC analysis was used to determine the presence of salts and other residues. Upon determination of yield, the DNA sample was sent for further analysis.

All DNA samples were swiped for radioactive contamination prior to analysis. Once cleared, samples were prepared for processing. Samples were dehydrated from 1 ml to 200 μl, combusted to CO₂ in individually sealed tubes, and the CO₂ was reduced to graphite on an iron or cobalt catalyst (Dingley, K. H., et al., (2003) DNA isolation and sample preparation for quantitation of adduct levels by Accelerator Mass Spectrometry. Mol. Tox. Prot.; in press). ¹⁴C analysis was conducted using AMS, a mass spectrometric method of detecting long-lived radioisotopes, with superior specificity and sensitivity. Most AMS measurements were done in 1 to 10 minutes, providing tens of thousands of counts for high precision. Notably, the AMS system of the invention allows use with liquid samples, such as DNA in solution. For data analysis, experimental ¹⁴C values were analyzed against radiocarbon bomb-spike measurements from previous geophysical analyses.

### Example 3

Other studies were performed for dating tooth enamel and wood. In one approach, tooth enamel was carefully chipped away to separate it from the underlying dentin, which undergoes continual cell turnover. Another protocol involved cutting away the crown of the tooth and subjecting the tooth to harsh chemical treatments that facilitate the removal of the dentin (Wieser, A., et al., (2001) Applied Radiation and Isotopes 54:793-799). This latter technique allows higher yields of pure enamel, and increases the accuracy of the dating procedure.

Using the AMS analysis of the invention, enamel from a 19 year old horse tooth was determined to be 14 years old, and enamel from a 6 year old horse was determined to be contemporary (FIG. 1I). These results were within the range expected, since the type of tooth subject to analysis does not emerge in horses until approximately 5 years of age. Horse teeth have been obtained from a Swedish animal slaughterhouse and human teeth obtained from a dental clinic and from the Karolinska Forensic Department. Teeth were obtained from the Karolinska Forensics Department, and the disclosed AMS analysis was used to assist in the identification of the age of the person by establishing the age of the ¹⁴C in the enamel of the person's teeth.

Data received from ¹⁴C analysis was used to produce delta ¹⁴C values (See, FIG. 1E). To determine a corresponding date for the delta ¹⁴C values, the bomb-spike curve was used. ¹⁴C values have been collected worldwide from a variety of sources, including atmospheric (at varying levels), tree ring, coral and oceanic sources. The most comprehensive bomb-spike curve was compiled by Ingeborg Levin (Levin, I., and B. Kromer, Twenty Years of Atmospheric 14CO2 observations at Schauinsland Station, Germany. Radiocarbon 39: 205 (1997); Levin, I., (1992) In: Radiocarbon after four decades: an interdisciplinary perspective. Taylor, R. E., Long, A., Kra, R. S., eds. New York: Springer-Verlag p503; Levin, I., et al., (1985) Radiocarbon 27:1-19), covering multiple sampling regions in the Northern and Southern Hemisphere from 1890 until 2000.

In accordance with this invention, data was collected to prepare a bomb-spike curve for Sweden, using tree rings from Swedish Pine. This project was initiated to ensure an accurate measurement of ¹⁴C exposure levels for the Swedish population over the last decade, or post-nuclear bomb testing. In addition, the data was used to provide information on current ¹⁴C levels, since no other information was available for 2000. Cross-sections from four Swedish pine trees were collected. For analysis, biopsies of wood are obtained from each ring, the wood is processed to remove all cellulose, the wood is combusted to CO₂, and the CO₂ is reduced to graphite on an iron or cobalt catalyst graphitizer. ¹⁴C analysis of the tree rings from the Swedish pine were in very good agreement with atmospheric ¹⁴C values published for the Northern Hemisphere (FIG. 1E)

### REFERENCES

Altman, J., and Das, G. D. Autoradiographic and histological evidence of postnatal hippocampal neurogenesis in rats (1965) J Comp Neurol 124(3):319-35.

Barnea, A., and Nottebohm, F. Seasonal recruitment of hippocampal neurons in adult free-ranging black-capped chickadees (1994) Proc. Natl. Acad. Sci. 91:11217-11221).

Barzilai, A., and Melamed, E. (2003) Molecular mechanisms of selective dopaminergic neuronal death in Parkinson's disease. Trends Mol. Med. 9(3):126-132.

Bernier, P. J., Bédard, A., Vinet, J., Lévesque, M., and Parent, A. Newly generated neurons in the amygdala and adjoining cortex of adult primates (2002) PNAS 99(17):11464-11469.

Czeh, B., Michaelis, T., Watanabe, T., Frahm, J., de Biurrun, G., van Kampen, M., Bartolomucci, A, and Fuchs, E. (2001) Stress-induced changes in cerebral metabolites, hippocampal volume, and cell proliferation are prevented by antidepressant treatment with tianeptine. Proc. Natl. Acad. Sci. USA 98(22): 12796-12801.

Dingley, K. H., Sánchez, E., Vogel, J. S., and Haack, K. W. (2003) DNA isolation and sample preparation for quantitation of adduct levels by Accelerator Mass Spectrometry. Mol Tox Prot.

Eriksson, P. S., Perfilieva, E., Björk-Eriksson, T., Alborn, A-M., Nordborg, C., Peterson, D. A., and Gage, F. H. (1998) Neurogensis in the adult human hippocampus. Nature Medicine 4(11):1313-1317.

Gould, E., Reeves, A. J., Fallah, M., Tanapat, P., Gross, C. G., and Fuchs, E. Hippocampal neurogenesis in adult Old World primates. (1999) Proc. Natl. Acad. Sci. USA 96:5263-5267.

Gould, E., Reeves, A. J., Graziano, M. S., and Gross, C. G. (1999) Neurogenesis in the neocortex of adult primates. Science 286:548-552.

Gould, E., Tanapat, P., McEwen, B. S., Flugge, G., and Fuchs, E. (1998) Proliferation of granule cell precursors in the dentate gyrus of adult monkeys is diminished by stress. Proc. Natl. Acad. Sci. USA 95:3168-3171.

Hamilton, M. L., Remmen, H. V., Drake, J. A., Guo, Z. M., Kewitt, K., Walter, C. A., and Richardson, A. Does oxidasive damage to DNA increase with age? (2001) PNAS 98(18):10469-10474.

Johansson, C. B., Momma, S., Clarke, D. L., Risling, M., Lendahl, U., and Fris'n, J. (1999) Identification of a neural stem cell in the adult mammalian central nervous system. Cell 96:25-34.

Koketsu, D., Mikami, A., Miyamoto, Y., and Hisatsune, T. Nonrenewal of neurons in the cerebral neocortex of adult Macaque monkeys (2003) J Neurosci 23(3):937-942.

Levin, I. (1992) In: Radiocarbon after four decades: an interdisciplinary perspective. Taylor, R. E., Long, A., Kra, R. S., eds. New York: Springer-Verlag p503.

Levin, I., and B. Kromer, Twenty Years of Atmospheric 14CO2 observations at Schauinsland Station, Germany. Radiocarbon 39: 205 (1997)

Levin, I., Kromer, B., Schoch-Fischer, H., (1985) 25 years of trophosperic ¹⁴C observations in central Europe. Radiocarbon 27:1-19.

Lewis, R. The neurobiology of rehabilitation (2003) The Scientist, 17(13):22-24.

Lovell, M. A., Robertson, J. D., Buchholz, B., Xie, C., and Markesby, W. R. Use of bomb pulse carbon-14 to age senile plaques and neurofibrillary tangles in Alzheimer's diseas (2002) Neurobiology of Aging 23:179-186.

McKay, R. Stem cells in the central nervous system (1997) Science 276:66-71.

Mok, H. Y. I., Druffel, E. R. M., and Rampone, W. M. Chronology of cholelithiasis (1986) New Eng J Med 314(17):1075-1077.

Nydal, R., and Lövseth, K. Tracing bomb 14C in the atmosphere, 1962-1980 (1983) J Geophysical Res 88:3621-3642.

Nydal, R., and Lövseth, K. 1997 Carbon-14 measurements in atmospheric CO₂ from northern and southern hemisphere sites, 1962-1993. Environmental Sciences Division, Office of Health and Environmental Research, U.S. Department of Energy. Publication No. 4582

Palmer, T. D., Takahashi, J., and Gage, F. H. (1997) The adult rat hippocampus contains primordial neural stem cells. Mol. Cell. Neurosci. 8:389-404.

Parent, J. M. (2003) Injury-induced neurogenesis in the adult mammalian brain. Neuroscientist 9(4):261-272.

Rakic, P. (2002) Neurogenesis in adult primate neocortex: an evaluation of the evidence. Nature Reviews 3:65-71.

Sambrook, J., Fritsch, E. F., and Maniatis, T. (1989) Molecular Cloning—A Laboratory Manual. 2nd Edition. Cold Spring Harbor Laboratory Press.

Santarelli, L., Saxe, M., Gross, C., Surget, A., Battaglia, F., Dulawa, S., Weisstaub, N., Lee, J., Duman, R., Arancio, O., Belzung, C., and Hen, R. (2003) Requirement of hippocampal neurogenesis for the behavioural effects of antidepressants. Science 301(8):805-809.

Serra, I., Avola, R., Condorelli, D. F., Surrentino, S., Renis, M., Kamiyama, M., Hashim, G. A., and Giuffrida, A. M. Acetylation and phosphorylation of histones and nonhistone chromosomal proteins in neuronal and glial nuclei purified from cerebral hemispheres of developing rat brain (1986) J Neurochem 46:1881-1887.

Spalding, K. L., Tan, M. M. L., Hendry, I. A., and Harvey, A. R. Anterograde transport and trophic actions of BDNF and NT-4/5 in the developing rat visual system (2002) MCN 19:485-500.

Struthers, L., Patel, R., Clark, J., and Thomas, S. Direct detection of 8-oxodeoxyguanosine and 8-oxoguanine by avidin and its analogues (1998) Anayltical Biochem 255:20-31.

Tatebayashi, Y., Lee, M. H., Li, L., Iqbal, K., and Grundke-Iqbal, I. (2003) The dentate gyrus neurogenesis: a therapeutic target for Alzeimer's disease. Acta Neuropathol. 105(3):225-232.

Wieser, A., EI. Faramawy, N., and Meckbach, R. (2001) Dependencies of the radiation sensitivity of human tooth enamel in EPR dosimetry. Applied Radiation and Isotopes 54:793-799.

Zhao, M., Momma, S., Delfani, K., Carlen, M., Cassidy, R., Johansson, C., Brismar, H., Shupliakov, O., Frisen, J., and Janson, (2003) A. M. Evidence for neurogenesis in the adult mammalian substantia nigra. Proc. Natl. Acad. Sci. USA 100(13):7925-7930.

1. A method for determining a birth date of a biomolecule comprising the steps of:
(a) providing a biomolecule;
(b) determining a delta ¹⁴C value of the biomolecule; and
(c) determining a birth date of said biomolecule by comparing the delta ¹⁴C value of said DNA with a calibration delta ¹⁴C chart to determine an birth date of said biomolecule.

2. The method of claim 1 wherein said biomolecule is whole cell tissue.

3. The method of claim 1 wherein said biomolecule is isolated from a tissue.

4. The method of claim 1 wherein said biomolecule is an animal, a plant, a virus or a part thereof.

5. The method of claim 1 wherein said biomolecule is isolated from a purified cell population.

6. The method of claim 5 wherein said purified cell population is a neuronal cell population.

7. The method of claim 1 wherein said biomolecule is a DNA molecule.

8. The method of claim 7 wherein said DNA molecule is isolated from a tissue, a cell line, or a purified cell population.

9. The method of claim 8 wherein said purified cell population is purified according to a secondary birth date sorting method.

10. The method of claim 9 wherein said secondary birth date sorting method is performed by fluorescence-activated cell sorter to separate different types of cells.
FACS sorting of cells based on histone acetylation level, DNA oxidation level, cellular lipofuschin level, or a combination thereof.

11. The method of claim 1 wherein said delta ¹⁴C value is determined by an accelerator mass spectrometer.

12. The method of claim 1 wherein said calibration delta ¹⁴C chart is selected from a calibration delta ¹⁴C chart shown in FIG. 1.

13. The method of claim 12 wherein said delta ¹⁴C chart is selected from the group consisting of a chart shown in FIG. 1A, FIG. 1B, FIG. 1C, FIG. 1D and FIG. 1E.

14. The method of claim 1, wherein said biomolecule is derived from a cell; and wherein said cell is analyzed by a secondary birth dating method before step (b).

15. The method of claim 14 wherein said secondary birth dating method comprises measuring histone acetylation level, DNA oxidation level, cellular lipofuschin level or a combination thereof.

16. The method of claim 14 wherein said secondary birth dating method comprise using a fluorescence activated cell sorter to measure said histone acetylation levels, DNA oxidation levels, cellular lipofuschin levels.

17. A method of determine the birth date of a biomolecule in a organism population comprising the steps of:
(a) collecting a sample of said biomolecule from an organism population;
wherein said biomolecule is purified away from other carbon containing molecules of said organism population;
(b) determining a delta ¹⁴C value of the carbon atoms in said biomolecule; and
(c) comparing the delta ¹⁴C value with a calibration delta ¹⁴C chart to determine a birth date of said biomolecule by

18. The method of claim 17 wherein said organism is an animal, a plant or a virus.

19. The method of claim 17 wherein said biomolecule is DNA.

20. The method of claim 17 wherein said biomolecule is tooth enamel from an animal.

21. The method of claim 20, wherein said animal is selected from the group consisting of a human, a horse, a pig, a cow, a rabbit, a dog, a rat and a mouse.

22. The method of claim 17, wherein said delta ¹⁴C value is determined by an accelerator mass spectrometer (AMS).

23. The method of claim 17 further comprising a step of calculating a birth date of said animal from the birth date of said biomolecule.

24. The method of claim 17 further comprising a step of measuring a second indicator of cell age.

25. The method of claim 24 wherein said second indicator is selected from the group consisting of histone acetylation levels, DNA oxidation levels, cellular lipofuschin levels or a combination thereof.

26. A method for determining an effect of a candidate agent on the cell proliferation of a tissue type in an animal comprising the steps of:
(a) determining a first birth date of a first cell sample from said tissue type from said animal using the method of claim 1;
(b) administering said candidate compound to said animal;
(c) determining a second birth date of a second cell sample from said tissue type animal using the method of claim 1; and
(d) comparing said first and second birth dates to determine if the candidate agent has an effect on cell proliferation.

27. The method of claim 26 wherein said tissue type is a CNS tissue type.

28. A method of determining the effect of a treatment on cell proliferation comprising the steps of:
(a) determining a first birth date of a first cell sample from an animal using the method of claim 1;
(b) inducing said event in said animal;
(c) determining a second birth date of a second cell sample from the same animal using the method of claim 1, wherein said first and second neuronal cell sample are from the same tissue; and
(d) comparing the first and second birth date to determine if the neurological event has an effect on cell proliferation.

29. The method of claim 28, wherein said treatment is selected from the group consisting of trauma, an induced disorder, a surgical procedure, and the administration of an agent.

30. The method of claim 28 wherein said treatment induces or affects a neurological disorder.

31. A method for determining a birth date of a biomolecule comprising the steps of:
(a) providing a biomolecule;
(b) determining an isotope concentration of said biomolecule;
(c) determining a birth date of said biomolecule by comparing the isotope concentration with a calibration isotope concentration chart to determine a birth date of said biomolecule.
